(19) United States
(12) Patent Application Publication
(10) Pub. No.: US 2009/0239783 A1
(43) Pub. Date: Sep. 24, 2009

(54) SYNERGISTIC EFFECTS OF COMBINED ADMINISTRATION OF MIRTAZAPINE AND A STIMULANT COMPOUND

(76) Inventor: Randal J. KIRK, Radford, VA (US)

Correspondence Address: DARBY & DARBY P.C., P.O. BOX 770, Church Street Station, New York, NY, 10008-0770, US

(21) Appl. No.: 12/479,340

(22) Filed: Jun. 5, 2009

Related U.S. Application Data
(63) Continuation of application No. 11/268,529, filed on Nov. 8, 2005.
(60) Provisional application No. 60/625,946, filed on Nov. 9, 2004.
(60) Provisional application No. 60/625,582, filed on Nov. 8, 2004.

(30) Foreign Application Priority Data
Nov. 7, 2005 (US) PCT/US2005/040268

(51) Int. Cl. A61K 38/02; A61K 31/55; A61P 25/24

(52) U.S. Cl. 514/2; 514/214.02

(57) ABSTRACT

The invention discloses combination therapies and formulations of a stimulant (e.g., amphetamine) and mirtazapine and their methods of use.

### CROSS REFERENCED AND RELATED APPLICATIONS

This application is a continuation application of U.S. patent application Ser. No. 11/268,529, filed Nov. 8, 2005, which claims priority to and the benefit of International Patent Application No. PCT/US2005/040268, filed Nov. 7, 2005; Provisional Patent Application No. 60/625,946, filed Nov. 9, 2004 and U.S. Provisional Patent Application No. 60/625,582 filed Nov. 8, 2004, each of which are hereby incorporated by reference in their entireties.

### BACKGROUND OF THE INVENTION

(i) Field of the Invention

The present invention relates to compounds, compositions, methods and uses of combinations of mirtazapine and a stimulant (e.g., amphetamine) for treatment of certain disorders.

(ii) Background of the Invention

Mirtazapine functions as an antidepressant or mood elevator and is used to treat a variety of disorders such as depression. Depression is a chronic illness that affects people of all ages. Although there are many effective antidepressant agents available, the current armamentarium of treatments is often not adequate, with unsatisfactory results in about one third of all subjects treated.

Mirtazapine has a tetracyclic chemical structure and belongs to the piperazino-azepine group of compounds. It is designated 1,2,3,4,10,14b-hexahydro-2-methylpyrazino [2,1-a] pyrido [2,3-c] benzazepine and has the empirical formula of C₁₇H₁₉N₃. The molecular weight of Mirtazapine is 265.36. Mirtazapine is a white to creamy white crystalline powder that is slightly soluble in water. Mirtazapine is supplied for oral administration as scored film-coated tablets containing 15 or 30 mg of mirtazapine, and unscored film-coated tablets containing 45 mg of mirtazapine. Each tablet also contains cornstarch, hydroxypropyl cellulose, magnesium stearate, colloidal silicon dioxide, lactose, and other inactive ingredients.

Mirtazapine is a presynaptic alpha-2 antagonist that has dual action by increasing noradrenergic and serotonergic neurotransmission. The enhancement of serotonergic neurotransmission is specifically mediated via 5-HT1 receptors because mirtazapine is a postsynaptic serotonergic 5-HT2 and 5-HT3 antagonist. In addition, mirtazapine has only a weak affinity for 5-HT1 receptors and has very weak muscarinic anticholinergic and histamine (H1) antagonist properties. Transient somnolence, hyperphagia and weight gain are the most commonly reported adverse events, which may be attributed to the antihistaminic (H1) activity of mirtazapine at low doses. Somnolence is the most commonly reported side effect of Mirtazapine. Mirtazapine also demonstrates important anxiolytic and sleep-improving effects, which may be related to its pharmacodynamic properties.

Stimulants, such as amphetamine, are prescribed for the treatment of various disorders, including attention deficit hyperactivity disorder (ADHD), obesity and narcolepsy. Stimulants such as amphetamine and methamphetamine stimulate the central nervous system and have been used medicinally to treat ADHD, narcolepsy and obesity.

### SUMMARY OF INVENTION

In one embodiment of the invention, a composition comprising mirtazapine and a stimulant (e.g., amphetamine) is provided. The composition may include mirtazapine and a stimulant (e.g., amphetamine) as the only pharmaceutically active components. Alternatively, other pharmaceutically active components are may be present in the composition. The composition preferably includes a pharmaceutically acceptable diluent, excipient, or carrier thereof. The composition may be provided in an oral dosage form such as a tablet, a capsule, a caplet, an oral solution, or an oral suspension. In a most preferred embodiment, the present invention exhibits a synergistic effect of treating one or more of the conditions described throughout the application while preventing drowsiness and somnolence.

In another embodiment, a composition comprising mirtazapine and a stimulant (e.g., amphetamine) may be provided wherein the stimulant is selected from amphetamine, methamphetamine, methylphenidate, or mixtures thereof. In another embodiment, the stimulant may include a covalently attached amino acid or amino acid containing compound. In another embodiment, the stimulant includes a covalently attached peptide. Preferably, the covalently attached peptide is attached to the stimulant through the C-terminus of the peptide.

In another embodiment, a composition comprising mirtazapine and a stimulant (e.g., amphetamine) is provided to a patient to treat depression, attention deficit hyperactivity disorder, attention deficit disorder, narcolepsy, obesity, and combinations thereof. In another embodiment, a composition comprising mirtazapine and a stimulant (e.g., amphetamine) is provided to a patient in need treatment with a stimulant (e.g., amphetamine). In another embodiment, a composition comprising mirtazapine and a stimulant (e.g., amphetamine) is provided to a patient in need of mirtazapine treatment.

In one embodiment, a method for the treatment of an mammal is provided that entails co-administering a therapeutically effective amount of mirtazapine or a pharmaceutically acceptable salt thereof (e.g. HCL, mesylate, etc.) and a therapeutically effective amount of a stimulant (e.g., amphetamine) or a pharmaceutically acceptable salt thereof (e.g. HCL, mesylate, etc.). In one aspect, the mammal is a human. Preferably the mammal is a human suffering from depression, attention deficit hyperactivity disorder, attention deficit disorder, narcolepsy, obesity, and combinations thereof. In one embodiment, the method of treatment entails administering a composition of mirtazapine and a stimulant (e.g., amphetamine) in an oral dosage form. The oral dosage form may be a tablet, a capsule, a caplet, an oral solution, or an oral suspension.

In one embodiment, a method for making a medicament is provided that comprises admixing mirtazapine or a pharmaceutically acceptable salt thereof (e.g. HCL, mesylate, etc.), a stimulant (e.g., amphetamine) or a pharmaceutically acceptable salt thereof (e.g. HCL, mesylate, etc.), and a pharmaceutically acceptable additive.

### DETAILED DESCRIPTION OF THE PREFERRED EMBODIMENTS

The invention is directed to co-administration of mirtazapine and a stimulant (e.g., amphetamine). In one embodiment, the co-administration of mirtazapine and a stimulant (e.g., amphetamine) is effective to treat depression, attention deficit hyperactivity disorder, attention deficit disorder, narcolepsy, obesity, and combinations thereof. In another embodiment, the invention exhibits a synergistic effect of treating one or more of the above conditions while preventing drowsiness and somnolence.

The following are exemplary stimulants Amphetamine (d-, 1-, and racemic), Benzphetamine, Caffeine, Diethylpropion, Mazindol, Methylphenidate (d-, 1-, and racemic), Phendimetrazine, Phentermine, Pemoline, and Sibutramine. Many of these exemplary stimulant compounds are currently marketed under the following tradenames and routes of administration: Adderall Oral, Adderall XR Oral, Adipex-P Oral, Alertness Aid Oral, Alertness Oral, Ammonia Aromatic Inhalation, Amoply Inhalation, Amphetamine-Dextroamphetamine Oral, Amphetamine Salt Combo Oral, Benzphetamine HCl Oral, Bontril PDM Oral, Bontril Slow Release Oral, Bontril Slow-Release Oral, Bontril SR Oral, Cafcit Injection, Cafcit Oral, Caffeine (Bulk) Miscell. (Med. Supl.; Non-Drugs), Caffeine Citrated Injection, Caffeine Citrated Miscell. (Med. Supl.; Non-Drugs), Caffeine Citrated Oral, Caffeine Citrate Miscell. (Med. Supl.; Non-Drugs), Caffeine-Ethyl Alcohol Oral, Caffeine Oral, Caffeine-Sodium Benzoate Injection, CONCERTA Oral, Cylert Oral, D-Amphetamine Sulfate (Bulk) Miscell. (Med. Supl.; Non-Drugs), Desoxyn Oral, Dexedrine Oral, Dexedrine Spansule Oral, Dexmethylphenidate HCl Oral, Dextroamphetamine Sulfate Oral, DextroStat Oral, Didrex Oral, Diethylpropion HCl Oral, Dopram Intravenous, Doxapram HCl Intravenous, Fastin Oral, Focalin Oral, Ionamin-15 Oral, Ionamin-30 Oral, Ionamin Oral, Keep Alert Oral, Kola Extract Oral, Kola Wine Oral, Lucidex Oral, Melfiat CR Oral, Meridia Oral, METADATE CD Oral, METADATE ER Oral, Methamphetamine HCl Oral, Methylin ER Oral, Methylin Oral, Methylphenidate HCl CR Oral, Methylphenidate HCl Oral, Modafinil Oral, No Doz Oral, Pemoline Oral, Phendimetrazine Tartrate Oral, Phentermine HCl Oral, Phentermine Resin Complex Oral, Pocion Jaccoud, Grandpa's Oral, Prelu-2 TR Oral, Pro-Fast HS Oral, Pro-Fast SA Oral, Pro-Fast SR Oral, Provigil Oral, Ritalin LA Oral, Ritalin Oral, Ritalin SR Oral, Sibutramine HCl Monohydrate Oral, Stay Awake Maximum Strength Oral, Stay Awake Oral, Tenuate Dospan Oral, Tenuate Oral, and Vivarin Oral.

As discussed above, an exemplary stimulant is amphetamine that will be discussed in further detail below. However, it will be appreciated that other stimulants, such as those listed above, may be used in combination with any of the embodiments disclosed herein. As used herein, “Amphetamine” shall mean any of the sympathomimetic phenethylamine derivatives that have central nervous system stimulant activity, such as but not limited to amphetamine (d-, 1-, and racemic), phentermine, methamphetamine, p-methoxyamphetamine, methylenedioxyamphetamine, 2,5-dimethoxy-4-methylamphetamine, 2,4,5-trimethoxyamphetamine and 3,4-methylenedioxymethamphetamine. Similarly, amphetamine also includes enantomers, racemic mixtures, and derivatives such as salts , prodrugs, and metabolites thereof. Suitable salts include acid addition salts, for example, hydrochloric, fumaric, maleic, citric, mesylate or succinic acid, these acids being mentioned only by way of illustration and without implied limitation.

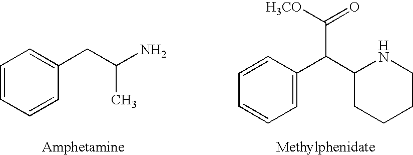

The term “amphetamine” used herein embraces modified amphetamine compounds wherein the amphetamine has been covalently bound to a chemical moiety. Thus, the composition of the present invention may be prepared using amphetamine compounds such as:
Lys-Amp=L-lysine-d-amphetamine, Lys-Amph, Lysine-Amphetamine, KAMP, K-amphetamine, or 2,6-diaminohexanoic acid-(1-methyl-2-phenylethyl)-amide
Phe-Amp=Phenylalanine-Amphetamine, FAMP, or
   2-amino-3-phenylpropanoic acid-(1-methyl-2-phenylethyl)-amide,
Ser-Amp=Serine-Amphetamine, SAMP, or
   2-amino-3-hydroxylpropanoic acid-(1-methyl-2-phenylethyl)-amide, Gly₃-Amp=GGG-Amphetamine, GGGAMP, or
   2-Amino-N-({[(1-methyl-2-phenyl-ethylcarbomyl)-methyl]-carbomyl}-methyl)-acetamide

### Prodrug Forms of Amphetamine

As further description of possible amphetamine conjugates that may be used as part of the invention are described in U.S. Pat. No. 10/857,619; U.S. Pat. No. 10/858,526 and PCT/US04/17204 each filed Jun. 1, 2004, each of which are incorporated by reference in its entirety.

Amphetamine may be attached to a chemical moiety. This prodrug chemical moiety would then be combined or co-administered with mertazapine or its derivative, analogs, salts thereof or combinations thereof. The chemical moieties may include any substance that results in a prodrug form, i.e., a molecule that is converted into its active form in the body by normal metabolic processes. The chemical moieties may be for instance, amino acids, peptides, glycopeptides, carbohydrates, nucleosides, or vitamins. The chemical moiety is covalently attached either directly or indirectly through a linker to the amphetamine. The site of attachment is typically determined by the functional group(s) available on the amphetamine.

In one embodiment of the invention, the chemical moiety is a carrier peptide as defined herein. The carrier peptide may be attached to amphetamine through the carrier's N-terminus, C-terminus or side chain of an amino acid which may be either a single amino acid or part of a longer chain sequence (i.e. a dipeptide, tripeptide, an oligopeptide or a polypeptide). Preferably, the carrier peptide is (i) an amino acid, (ii) a dipeptide, (iii) a tripeptide, (iv) an oligopeptide, or (v) polypeptide. The carrier peptide may also be (i) a homopolymer of a naturally occurring amino acid, (ii) a heteropolymer of two or more naturally occurring amino acids, (iii) a homopolymer of a synthetic amino acid, (iv) a heteropolymer of two or more synthetic amino acids, or (v) a heteropolymer of one or more naturally occurring amino acids and one or more synthetic amino acids. A further embodiment of the carrier and/or conjugate is that the unattached portion of the carrier/conjugate may be in a free and unprotected state. Preferably, synthetic amino acids with alkyl side chains are selected from alkyls of C₁-C₁₇ in length and more preferably from C₁-C₆ in length.

In one embodiment, the amphetamine is attached to a single amino acid which is either naturally occurring or a synthetic amino acid. In another embodiment, the amphetamine is attached to a dipeptide or tripeptide, which could be any combination of the naturally occurring amino acids and synthetic amino acids.

In another embodiment, the side chain attachment of amphetamine to the polypeptide or amino acid are selected from homopolymers or heteropolymers of glutamic acid, aspartic acid, serine, lysine, cysteine, threonine, asparagine, arginine, tyrosine, and glutamine. Examples of peptides include, Lys, Ser, Phe, Gly-Gly-Gly, Leu-Ser, Leu-Glu, homopolymers of Glu and Leu, and heteropolymers of (Glu)ₙ-Leu-Ser. In a preferred embodiment, the composition is selected from Lys-Amp, Ser-Amp, Phe-Amp, and Gly-Gly-Gly-Amp.

In another embodiment, the invention provides a carrier and amphetamine that are bound to each other but otherwise unmodified in structure. This embodiment may further be described as the carrier having a free carboxy and/or amine terminal and/or side chain groups other than at the location of attachment for the amphetamine. In a preferred embodiment, the carrier, whether a single amino acid, dipeptide, tripeptide, oligopeptide or polypeptide, comprises only naturally occurring amino acids. For additional methods of attaching amphetamine to carriers, see application number U.S. patent application Ser. No. 10/156,527, and/or PCT/U.S.03/05524 and/or PCT/U.S.03/05525 each of which is hereby incorporated by reference in its entirety.

The term “mirtazapine” as used herein includes the compound 1,2,3,4,10,14b-hexahydro-2-methylpyrazino [2,1-a] pyrido [2,3-c] benzazepine, enantomers thereof, racemic mixtures thereof, derivatives such as salts, prodrugs, and metabolites thereof. Suitable salts include acid addition salts, for example, hydrochloric, fumaric, maleic, citric or succinic acid, these acids being mentioned only by way of illustration and without implied limitation. Although the term “mirtazapine” may embrace certain related compounds, it always includes the compound 1,2,3,4,10,14b-hexahydro-2-methylpyrazino [2,1-a] pyrido [2,3-c] benzazepine. Mirtazapine has the following chemical structure:

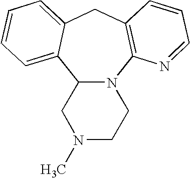

Mirtazapine may be prepared as disclosed in U.S. Pat. No. 4,062,848 to van der Burg. It will be appreciated that mirtazapine contains a centre of chirality. The present invention includes each of the individual (R) and (S) enantiomers of mirtazapine and it salts in a form substantially free from the other enantiomer (i.e. having an enantiomeric purity of greater than 95% and preferably greater than 99%), as well as mixtures of the enantiomers in any proportion including a racemic mixture.

Further, mertazapine its derivatives and its uses are described for instance in U.S. Pat. Nos. 6,667,297; 6,780,860; 6,774,230; 6,723,845; 6,667,297; 6,660,730; 6,649,605; 6,627,653; 6,589,556; 6,576,764; 6,552,189; 6,552,014; 6,545,149; 6,541,043; 6,495,154; 6,489,341; 6,482,440; 6,437,120; 6,420,351; 6,403,597; 6,399,310; 6,303,595; 6,281,207; 6,228,875; 6,211,171; 6,150,353; 6,114,324; 6,040,301; 5,977,099; 5,922,341; 5,208,261; 5,082,864 4,062,848 each of which is hereby incorporated by reference in their entirety for both their disclosure of mertazapine derivatives and uses of mertazapine. In general, a suitable dose of mirtazapine or a pharmaceutically acceptable salt thereof for administration to a human will be in the range of 0.01 to 30 mg per kilogram body weight of the recipient per day, preferably in the range of 0.1 to 5 mg per kilogram body weight per day and most preferably in the range of 0.3 to 1.0 mg per kilogram body weight per day. In one embodiment the standard dosage of mirtazapine may be low than typically administered due to the synergistic effects of co-administration with amphetamine.

In another embodiment of the invention, a composition including amphetamine and mirtazapine is adapted for oral administration. Optionally, said composition may be in the form of a tablet, capsule, oral solution, oral suspension, or other oral dosage form discussed herein. In the methods of the present invention, a composition of amphetamine and mertazapine is preferably administered orally.

In another embodiment, at least one of the drugs of interest (e.g. amphetamine and mertazapine) may further comprise a polymer blend that comprises a hydrophilic polymer and/or a water-insoluble polymer. The polymers may be used according to industry standards to further enhance the sustained release/abuse resistant properties of the amphetamine conjugate without reducing the abuse resistance. For instance, a composition might include: about 70% to about 100% amphetamine conjugate by weight, from about 0.01% to about 10% of a hydrophilic polymer (e.g. hydroxypropyl methylcellulose), from about 0.01% to about 2.5% of a water-insoluble polymer (e.g. acrylic resin), from about 0.01% to about 1.5% of additives (e.g. magnesium stearate), and from about 0.01% to about 1% colorant by weight. The above amounts are only exemplary. For instance an alternative formulation may include: the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Hydrophilic polymers suitable for use in the sustained release formulations include one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art, or a combination of such polymers. These hydrophilic polymers gel and would dissolve slowly in aqueous acidic media thereby allowing the amphetamine conjugate to diffuse from the gel in the stomach. When the gel reaches the intestines it would dissolve in controlled quantities in the higher pH medium to allow further sustained release. Preferred hydrophilic polymers are the hydroxypropyl methylcelluloses such as those manufactured by The Dow Chemical Company and known as Methocel ethers, such as Methocel E10M.

Other formulations may further comprise pharmaceutical additives including, but not limited to: lubricants such as magnesium stearate, calcium stearate, zinc stearate, powdered stearic acid, hydrogenated vegetable oils, talc, polyethylene glycol, and mineral oil; colorants such as Emerald Green Lake, FD&C Red No. 40, FD&C Yellow No. 6, D&C Yellow No. 10, or FD&C Blue No. 1 and other various certified color additives (See 21 CFR, Part 74); binders such as sucrose, lactose, gelatin, starch paste, acacia, tragacanth, povidone polyethylene glycol, Pullulan and corn syrup; glidants such as colloidal silicon dioxide and talc; surface active agents such as sodium lauryl sulfate, dioctyl sodium sulfosuccinate, triethanolamine, polyoxyethylene sorbitan, poloxalkol, and quarternary ammonium salts; preservatives and stabilizers; excipients such as lactose, mannitol, glucose, fructose, xylose, galactose, sucrose, maltose, xylitol, sorbitol, chloride, sulfate and phosphate salts of potassium, sodium, and magnesium; and/or any other pharmaceutical additives known to those of skill in the art. In one preferred embodiment, a sustained release formulation further comprises magnesium stearate and Emerald Green Lake.

An amphetamine conjugate, which is further formulated with excipients, may be manufactured according to any appropriate method known to those of skill in the art of pharmaceutical manufacture. For instance, the amphetamine-conjugate and a hydrophilic polymer may be mixed in a mixer with an aliquot of water to form a wet granulation. The granulation may be dried to obtain hydrophilic polymer encapsulated granules of amphetamine-conjugate. The resulting granulation may be milled, screened, then blended with various pharmaceutical additives such as, water insoluble polymers, and/or additional hydrophilic polymers. The formulation may then tableted and may further be film coated with a protective coating which rapidly dissolves or disperses in gastric juices.

However, it should be noted that the amphetamine conjugate controls the release of amphetamine into the digestive tract over an extended period of time resulting in an improved profile when compared to immediate release combinations and therefore does not require addition release matrices. As such it is possible to mix the mertazapine according to industry standards and either co-administer an amphetamine conjugate with mertazapine or at the final stages of mixing blend or compress an amphetamine conjugate with a delayed and/or immediate release mertazapine. In a preferred embodiment, no further sustained release additives are required to achieve a blunted or reduced pharmacokinetic curve (e.g., reduced euphoric effect) while achieving therapeutically effective amounts of amphetamine release when taken orally.

The compounds of the invention can be administered by a variety of dosage forms. Any biologically-acceptable dosage form known to persons of ordinary skill in the art, and combinations thereof, are contemplated. Examples of preferred dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets and combinations thereof.

The most effective means for delivering the abuse-resistant compounds of the invention is orally, to permit maximum release of the amphetamine, and provide therapeutic effectiveness and/or sustained release while maintaining abuse resistance. When delivered by oral route the amphetamine is released into circulation, preferably over an extended period of time as compared to amphetamine alone.

Formulations of the invention suitable for oral administration can be presented as discrete units, such as capsules, caplets or tablets. These oral formulations also can comprise a solution or a suspension in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube of a patient who is unable to swallow.

Soft gel or soft gelatin capsules may be prepared, for example by dispersing the formulation in an appropriate vehicle (vegetable oils are commonly used) to form a high viscosity mixture. This mixture is then encapsulated with a gelatin-based film using technology and machinery known to those in the soft gel industry. The industrial units so formed are then dried to constant weight.

Chewable tablets, for example may be prepared by mixing the formulations with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are versed in the processes and the machinery used as the chewable dosage form is a very common dosage form in the pharmaceutical industry.

Film-coated tablets, for example may be prepared by coating tablets using techniques such as rotating pan coating methods or air suspension methods to deposit a contiguous film layer on a tablet.

Compressed tablets, for example may be prepared by mixing the formulation with excipients intended to add binding qualities to disintegration qualities. The mixture is either directly compressed or granulated then compressed using methods and machinery known to those in the industry. The resultant compressed tablet dosage units are then packaged according to market need, i.e., unit dose, rolls, bulk bottles, blister packs, etc.

The invention also contemplates the use of biologically acceptable carriers that may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

Preferred plasticizers may be selected from the group consisting of diethyl phthalate, diethyl sebacate, triethyl citrate, cronotic acid, propylene glycol, butyl phthalate, dibutyl sebacate, castor oil and mixtures thereof, without limitation. As is evident, the plasticizers may be hydrophobic as well as hydrophilic in nature. Water-insoluble hydrophobic substances, such as diethyl phthalate, diethyl sebacate and castor oil are used to delay the release of water-soluble vitamins, such as vitamin B6 and vitamin C. In contrast, hydrophilic plasticizers are used when water-insoluble vitamins are employed which aid in dissolving the encapsulated film, making channels in the surface, which aid in nutritional composition release.

It should be understood that in addition to the ingredients particularly mentioned above, the formulations of this invention can include other suitable agents such as flavoring agents, preservatives and antioxidants. Such antioxidants would be food acceptable and could include vitamin E, carotene, BHT or other antioxidants known to those of skill in the art.

Other compounds which may be included by admixture are, for example, medically inert ingredients, e.g., solid and liquid diluent, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Liquid dispersions for oral administration may be syrups, emulsions or suspensions. The syrups may contain as carrier, for example, saccharose or saccharose with glycerol and/or mannitol and/or sorbitol. The suspensions and the emulsions may contain a carrier, for example a natural gum, agar, sodium alginate, pectin, methylcellulose, carboxymethylcellulose or polyvinyl alcohol.

The dose range for adult human beings will depend on a number of factors including the age, weight and condition of the patient. Tablets and other forms of presentation provided in discrete units conveniently contain a daily dose, or an appropriate fraction thereof, of one or more of the compounds of the invention. For example, units may contain from 5 mg to 500 mg, but more usually from 10 mg to 250 mg, of one or more of the compounds of the invention. It should also be appreciated that due to the synergistic effects of mertazapine and amphetamine that a low dosage of one or both may be used.

It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period. The doses may be uneven doses with regard to one another or with regard to the individual components at different administration times.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may additionally include an indication of the above specified time periods for administering the compositions. For example, the indicia may be time indicia indicating a specific or general time of day for administration of the composition, or the indicia may be a day indicia indicating a day of the week for administration of the composition. The blister pack or other combination package may also include a second pharmaceutical product.

It should be understood that each of the above recited embodiments may be used in individually or in combination with amphetamine or mirtazapine to achieve synergistic treatment and dosage forms.

What is claimed is:

1. A composition comprising a stimulant or a derivative or analog thereof and mirtazapine or a derivative or analog thereof.

2. A composition of claim 1, consisting essentially of a stimulant or a derivative or analog thereof and mirtazapine or a derivative or analog thereof.

3. The composition of claim 1, further comprising a pharmaceutically acceptable diluent, excipient, or carrier thereof.

4. The composition of claim 3 in an oral dosage form.

5. The composition of claim 4, wherein said oral dosage form is a tablet, a capsule, a caplet, an oral solution, or an oral suspension.

6. The composition of claim 5, wherein said stimulant includes a covalently attached amino acid or amino acid containing compound.

7. The composition of claim 6, wherein said stimulant includes a covalently attached peptide.

8. The composition of claim 7, wherein said peptide is attached to the stimulant compound through the C-terminus of the peptide.

9. The composition of claim 8, wherein said stimulant is amphetamine covalently bound to lysine.

10. The composition of claim 9, wherein said stimulant is amphetamine, phentermine, methamphetamine, methylphenidate, or mixtures thereof.

11. A method of treating attention deficit hyperactivity disorder comprising providing a patient with the composition according to claim 1.

12. A method of treating depression comprising providing a patient with the composition according to claim 7.

13. A method for treating a patient in need of a stimulant comprising providing to a patient a composition according to claim 1.

14. A method for treating a patient in need of mirtazapine comprising providing to a patient a composition according to claim 1.

15. A method for the treatment of an animal, comprising co-administering a therapeutically effective amount of mirtazapine or a derivative or analog thereof or a pharmaceutically acceptable salt thereof and a therapeutically effective amount of stimulant or a derivative or analog thereof, or a pharmaceutically acceptable salt thereof.

16. The method according to claim 15, wherein the animal is a human.

17. The method according to claim 16, wherein the treatment is performed on a human suffering from depression, attention deficit hyperactivity disorder, attention deficit disorder, narcolepsy, obesity, and combinations thereof.

18. A method for making a medicament, comprising admixing mirtazapine or a derivative or analog thereof or a pharmaceutically acceptable salt thereof, a stimulant or a derivative or analog thereof or a pharmaceutically acceptable salt thereof, and a pharmaceutically acceptable additive.

19. A method according to claim 17, where said stimulant is amphetamine, phentermine, methamphetamine, methylphenidate, or mixtures thereof.
